(19) United States
(12) Patent Application Publication
(10) Pub. No.: US 2020/0022895 A1
(43) Pub. Date: Jan. 23, 2020

(54) TISSUE EXPANSION METHOD

(71) Applicant: The Medical Research, Infrastructure, and Health Services Fund of the Tel Aviv Medical Center, Tel Aviv (IL)

(72) Inventors: Amir INBAL, Tel Aviv (IL); Andrew GREENSMITH, Camberwell (AU); Nir SHANI, Ramat Hasharon (IL)

(73) Assignee: The Medical Research, Infrastructure, and Health Services Fund of the Tel Aviv Medical Center, Tel Aviv (IL)

(21) Appl. No.: 16/039,427

(22) Filed: Jul. 19, 2018

(51) Int. Cl. A61K 8/73; A61K 8/04; A61K 9/00; A61L 27/36; A61Q 19/00

(52) U.S. Cl. CPC A61K 8/735; A61K 8/042; A61Q 19/00; A61L 27/36; A61K 9/0019

(57) ABSTRACT

The invention provides a method for skin expansion utilizing at least one polymer.

### TECHNOLOGICAL FIELD

The current invention generally concerns a method for tissue expansion utilizing a minimally invasive method involving use of polymeric materials.

### BACKGROUND ART

Tissue expansion is a reliable method for providing additional cutaneous tissue, for a given reconstructive effort. The effects of tissue expansion on skin include increased surface area and vascularity and thus allow coverage of a variety of complex open wounds.

The technique of tissue expansion is more than three decades old and has been extensively used in reconstructive surgery in various body parts. Nevertheless, tissue expansion still requires vigilant patient selection, careful planning and faultless execution to succeed and the process typically lasts for more than 8-12 weeks and involves two sittings of surgery [11.

To date, the process of tissue expansion involves three steps. The first step is a surgical step where a balloon-like prosthesis (expander) is inserted under the skin to be expanded. Next, a few months following surgery, the patient undergoes expansion at consecutive visits at the clinic by saline injections via a port attached to the expander. Last, a second surgery in which the tissue expander is removed and final reconstruction is achieved based on the additional tissue expanded and gained.

Several types of tissue expander devices exist, based on shape, size and type of filling valve. They can be standard, customized and anatomic to the donor site (breast) and may be provided in a wide range of sizes, varying according to the anatomic site (100 to 2000 ml). Saline is delivered in a controlled fashion via the valve port, which is either integrated into the prosthesis or connected to the device by silicone tubing of customized length. An integrated system offers the advantage of undermining a single pocket for expander placement, but also place the implant at risk of perforation by a misplaced needle. Internal remote ports remove the danger of perforation, but introduce the potential complications of overturn of the port, tube obstruction and migration.

Prosthetic reconstruction utilizing a 2-stage saline tissue expander-to-implant procedure is a common technique for breast reconstruction, being used for nearly 50 years. Only in December 2016, a carbon dioxide-filled, remote-controlled tissue expander received U.S. Food and Drug Administration clearance. This tissue expander, known as the AeroForm Tissue Expander System (AirXpanders, Inc., Palo Alto, Calif.), is a patient-controlled, needle-free expander operated by a wireless remote-control device, which allows patients to perform home expansion, precluding the need for percutaneous saline injections. However, implant extrusion and under-expansion due to CO₂ permeation, are potential complications, as well as the added surgical risks to insert the expander.

The need for an additional surgical stage has potential for skin necrosis [2].

The use of Hyaluronic acid (HA) in cosmetic and dermatological applications is widely known and HA is well tolerated and no immunogenicity is associated with its use. Following its administration, HA is subject to degradation through different pathways (e.g. enzymatic, temperature, free radicals), and therefore, its longevity in vivo is limited [3].

HA based dermal fillers have been widely used as soft tissue filler augmentation agents for treating facial wrinkles, fine lines, scars and deep tissue folds [4]. HA has been shown to possess a relative ease of application, minimal downtime, reversibility and little adverse effects [5].

Large particle HA is a gel used for volume restoration of soft tissues. Large particle HA filler injections have been shown to be a predictable, safe, and long-lasting non-surgical procedure to fill contour defects that arise after liposuction and represent a good option for patients who refuse to undergo an additional surgery to fill the arisen skin depressions [5].

However, while several HA fillers are widely approved for injection into mid-dermis to deep dermis for correction of moderate to severe wrinkles and folds, as well as for volume augmentation; their use as an expansion modality was never practiced.

### REFERENCES

[1] Milind S, et al., Indian J Plast Surg. 2013 May-August; 46(2): 333-348.
[2] Jung C and Hong-Xing Z; Plast Reconst Surg, 207, 119(6), pp:1759-1766.
[3] Philipp-Dormston W G; Hautarzt. 2018; 69(6):491-509.
[4] Gold H M, Clin Intery Aging. 2007 September; 2(3): 369-376.
[5] Paliwal S, et al., Plast Reconstr Surg, 2014; 134(6): 1224-1233.

### GENERAL DESCRIPTION

To date, traditional tissue expansion has failed to provide a mainstream tool for skin reconstruction, especially where small organs such as the ear and fingers are concerned; hence a viable method for skin reconstruction is needed.

The inventors of the present invention have developed a method for skin expansion utilizing a polymeric material, such as hyaluronic acid (HA), that excludes the need for a surgical procedure or the insertion of a prosthetic to achieve skin expansion. Thus, the method of the invention renders moot the need to insert an expansion device to achieve expansion and subsequent skin growth, hence reduces risk of skin rupture and necrosis. This drastically reduces the morbidity involved in tissue expansion.

The herein described method constitutes a cost-effective method for minimally invasive tissue expansion using highly cohesive filler formulation, allowing device- or prosthetic-free skin reconstruction. Tissue expansion with a polymer such as HA completely obviates the need for the first surgical step mentioned above. Injections of a filler formulation comprising the polymer, e.g., HA, can be simply tailored by the surgeon, avoiding the physical restrictions of a custom-made expander and reducing costs involved with subject-specific tissue expander devices. The device-free tissue expansion method of present invention further circumvents inherent complications that plague the standard skin expansion method, such as potential vascular problems, port problems, expander extrusion and deflation issues.

The herein described methods and HA expansion kit are particularly suitable for obtaining skin expansion in very delicate areas of the face, limb and fingers where a usual expander device is contraindicated.

Thus, in a first aspect, there is provided a method of skin expansion, the method comprising injecting an amount (or volume) of a filler formulation at a skin region, said amount being sufficient to cause expansion and growth of skin at said skin region over time, and optionally repeating said injection one or more times, to thereby cause expansion and growth of said skin region.

In some embodiments, the method further includes a step of manipulating said grown skin region to enable use of the skin region in a method of reconstructive treatment or surgery.

In accordance with the invention, the method is free of use of any solid or semisolid expansion device or prosthesis that is placed under the skin region.

As noted herein, the injection of the filler formulation occurs at a skin region and at a skin depth that is sufficient to bring about lifting of the skin, and which at the same time does not result in any toxicity. The injection is does non-systemic and is made to a region under a skin tissue that is predetermined based, inter alia, on the nature of the filler formulation, e.g., the type of polymer used, the degree of skin expansion required, the therapeutic or cosmetic purpose, etc.

Cosmetic skin augmentation is excluded from purposes achievable by methods of the invention. In other words, methods of the invention are not intended to achieve permanent or time-limited skin augmentation. As used herein, ‘skin augmentation’ is or involves treatment and/or correction of conditions selected from scares (e.g. scares caused by acne and facial scares), wrinkles (e.g. correction of facial lines), furrows, frown lines (glabellar lines) smoker's lines (perioral lines), marionette lines (oral commissures), worry lines (forehead lines), crow's feet (periorbital lines), deep smile lines (nasolabial furrows), smile lines (nasolabial lines), cheek depressions, lip enhancement, witch's chin folds (chin augmentation) and other defects such as enhancement or filling in of certain specific facial features such as the lips or chin, depressed scars in the skin, at any body region, yet particularly in the forehead, around the eyes, nose and lips.

Thus, skin augmentation excluded from the present invention generally refers to an injection or multiple injections (e.g. of various filling agents) into the skin that do not result in sufficient/substantial lifting capacity as defined herein, i.e. does not lead to a lifting of the skin region above the skin surface level prior to injection, and/or does not lead to a skin expansion of more than 2 mm/cm of the skin as compared to (e.g. resting) skin prior to the injection.

Methods of the invention intend to achieve an expansion and subsequent growth of a skin tissue that may be subsequently manipulated, as known in the art, e.g., partially or fully harvested or extended over a skin defect. To achieve skin expansion and skin growth, the filler formulation must impose lifting of the skin at the skin region while maintaining continuous skin tension, which may increase over time. Thus, methods of the invention should involve delivery of a filler formulation of a selected volume, consistency or composition to impose a suitable skin lifting capacity. The “lifting capacity” generally refers to the effect of the filler formulation imposed on the skin as reflected in the upward lifting of the skin in a direction generally perpendicular to the skin surface. Thus, in accordance with the present invention, sufficient/substantial lifting capacity that can lead to skin expansion is defined as lifting of at least 2, 3,4, 5, 6, 7, 8, 9, 10 mm/cm of the skin as compared to the skin region at a resting state, namely prior to the injection.

In some embodiments, the skin lifting needed to achieve expansion and growth of the skin tissue depends on the degree of extension and growth needed. In some embodiments, the skin is lifted by between 5 and 10, 10 and 20, 20 and 30, 30 and 40, 40 and 50, 50 and 60, 60 and 70, 70 and 80, 80 and 90 or 90 and 100 mm from the skin position prior to the injection.

Thus, “skin expansion” may be defined as an expansion of a skin region that directly causes the biological growth of skin as measured after removing the filler formulation from the skin region (e.g. by aspiration/suction). Accordingly, the surface area of the skin following removal of at least 10, 20, 30, 40, 50, 60, 70, 80, 90, 95 or 99 percent of the filler formulation from the body is larger by at least 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190% or higher, as compared with the surface area of the skin before injection of the filler formulation.

Without wishing to be bound by theory, the injection of the filler formulation leads to mechanical (elastic) stretching of the skin. When skin is stretched beyond its physiological limit, mechanotransduction pathways are activated leading to the biological growth of the skin (involving e.g. increased cellular growth, collagen synthesis and formation of new skin cells). As readily understood by a person of skill in the art, the skin expansion caused by stretching of skin may be followed by skin relaxation (e.g. retraction to the approximate skin size present prior to the injection) such that at least one cycle of stretching and relaxation may be required to trigger biological skin growth, as defined herein. The biological skin growth which follows skin expansion caused by injection of the filler formulation can occur after a single injection of the filler formulation or after two or more such injections and depends on various parameters such as the location of the injection, the type/viscosity of filler formulation used for the injection, the amount injected, the condition of the patient etc.

Thus, wherein injection in some skin locations may lead to skin growth after a single injection, injecting the filler formulation into other skin areas may require the injection to be carried out periodically, e.g. over days or weeks for sufficient stretching/over-stretching to take place that could, in turn, lead to biological skin growth.

The biological skin growth resulting from skin expansion, as defined herein, may be uniform, e.g. occur at a relatively constant rate following each injection of filler formulation, yet in some cases can also be non-uniform, e.g. occur at non-constant rates after and between injections of the filler formulation. Thus, for example, biological growth can begin only after a few (e.g. 5) injections or injection sessions (wherein each session involves a single or more injections) and decrease or increase thereafter.

In some embodiments, the biological growth of skin, as described herein, is permanent. In other embodiments, the biological growth of skin, as described herein, retracts by 1, 2, 3, 4, 5, 6, 7, 8, 9, or 10% or more upon removal of the filler formulation or after a period of time.

The “filler formulation” used in accordance with the invention is a liquid or gel or semi-gel composition that is suitable for injection, and comprising at least one polymeric material suitable for injection into a skin tissue of the subject. The at least one polymeric material is an inert material that does not substantially interact with the tissue into which it is injected. The formulation may be in a form selected from gels, dispersions, solutions, suspensions, slurries and mixtures thereof. The formulation is, in some embodiments, in the form of a gel or a gel-like form, optionally in the shape of particles or containing particles. The particles may be 1 to 5 mm in size, optionally dispersed in a carrier that may be selected from physiologically stable buffers, aqueous media or gels.

The at least one polymeric material may be synthetic, semi-synthetic or naturally-derived. It may be a single polymeric material or a mixture of two or more such materials. In some embodiments, the at least one polymer is a biopolymer or a polysaccharide.

In some embodiments, the at least one polymeric material is selected from poly-L-lactic acid, polymethylmethacrylate (PMMA), calcium hydroxyapatite (CaHA), chitosan, alginate and hyaluronic acid (HA), or any mixture of two or more such polymers. In some embodiments, the at least one polymer is formed of a chemically associated pre-polymer of any of the aforementioned polymers or is formed by chemically associating two or more polymers selected from the aforementioned polymers.

A filler formulation may comprise a polymer concentration that is at least 5 mg/ml, or is between 5 and 50, 5 and 100, 10 and 50, 10 and 100, 20 and 50, 20 and 100, 30 and 50, 30 and 100 mg/ml.

In some embodiments, the at least one polymer material is HA. In formulations comprising HA, the HA concentration may be at least 5 mg/ml, or is between 5 and 50, 5 and 100, 10 and 50, 10 and 100, 20 and 50, 20 and 100, 30 and 50, 30 and 100 mg/ml.

In some embodiments, the at least one polymeric material may be in the form of a phase transfer material, e.g., that can be injected in a liquid or semi-liquid form (or a gel form) and which undergo solidification or a viscosity change into a harder, less flowing material.

The filler formulation may be formed into material particles, e.g., gel particles having a size supporting skin lifting capacity capabilities. In some embodiments, the at least one polymeric material is provided in particulate form.

In some embodiments, the filler formulation is in the form of material particles of between about 500 and 2,000, 800 to 1,500, or 900 to 1,200 particles/ml.

In some embodiments, the filler formulation is in the form of a gel material.

The at least one polymeric material may be used in a natural form, as salts thereof (e.g. sodium, potassium, magnesium and calcium salts), modified forms thereof, crosslinked forms thereof, copolymers thereof, block polymers or block co polymers thereof, or mixtures thereof. In addition to the at least one polymeric material, the filler formulation may further comprise at least one carrier material and further optionally at least one excipient.

In some embodiments, the at least one excipient is selected from the group consisting of a vitamin (e.g. C, E, B), a degradation inhibitor (e.g. chondroitin sulphate), an antioxidant, a stabilizer, a steroid, a retinoid or salt/derivative thereof. The excipient may be alternatively or additionally selected to increase the stability (e.g. from heat and oxidation) and longevity of the filler formulation, for example by providing protection from chemical degradation.

In some embodiment, the at least one polymer is hyaluronic acid (HA), which may be synthetic, semisynthetic or commercially obtained. As known in the art, HA is a naturally occurring, water soluble polysaccharide having a molecular weight range of about between 6×10⁴ and 8×10⁶ Da. Commercially available HA and cross-linked HA may be prepared according to methods known in the art, for example as disclosed in U.S. Pat. Nos. 5,356,883 and 6,013,679, each of which being incorporated herein by reference.

In some embodiments, the HA is selected amongst commercially available HAs such as those having the trade-names Macrolane®, Hylaform®, Hylaform® Plus, Restylane®, Perlane®, Restylane® Fine Lines, Hylaform® Plus, Captique™, Juvederm™ Ultra, Juvederm™ Ultra plus, Puragen™ Plus, HYAcorp MLF1® and Belotero.

In one embodiment, the HA in the filler formulation is Macrolane®.

In some embodiments, the filler formulation comprises cross linked HA such as HA formed by linking two or more individual HA molecules, either directly or by a linking moiety or HA cross linked with other polymers or low molecular weight substances.

In some embodiment, the filler formulation is sterilized, e.g. by autoclaving, wherein the sterilized formulation is stable at ambient temperature (e.g. for at least 6 months).

As readily understood by the person of skill in the art, the herein defined filler formulation does not contain constituents which may elicit an allergic response and is stable, well-tolerated, grossly homogeneous, syringable and contains only pharmaceutically acceptable additives and excipients.

In accordance with the present invention, the filler formulation has suitable viscoelasticity (e.g. as measured by G* [“hardness”], G′ [elastic properties], G″ [viscous properties] and Tan δ [ratio between viscous and elastic properties] to provide a desirable skin lifting capacity and is configured to withstand different types of deformation and forces when injected in various skin areas.

In some embodiments, the G′ value of the filler formulation is between about 10 and about 1000 Pa.

In some embodiments, the G″ value of the filler formulation is between about 20 and about 500 Pa.

In some embodiments, the Tan δ value of the filler formulation is between about 0.05 and 1.

As the person versed in the art readily understands, a combination of various factors may dictate the skin lifting capacity and duration thereof of the filler formulation. For example, by controlling at least one of the following parameters and/or combinations thereof, the medical practitioner can control the skin lifting capacity and consequently the effectiveness of the treatment of the skin defect.

Some non-limiting examples of such parameters are:
injection frequency of filler formulation (e.g. daily, once weekly etc.);
volume (cc) of each injection;
particle size of filler formulation (e.g. HA gel particle size);
viscoelasticity of filler formulation;
cohesivity of filler formulation (i.e. behavior of filler formulation after injection into skin);
needle gauge;
extrusion force (i.e. measure of the force needed to inject the filler formulation at a fixed rate through the needle).

The volume of the formulation that may be injected may be delivered by a single injection or by one or more or sequential injections. As used herein, the term “sequential injections” generally refers to a series of injections into the skin tissue (e.g. into the same of different locations, or adjacent to the periphery of a skin defect), performed over the course of a few days, weeks or months, wherein the amount of the filler formulation may or may not be increased from one injection event to the next. The sequential injections result in a continuous increase in the volume of filler formulation at the site of injection such that a continuous expansion of the skin is achieved over time. It should be noted that injections of filler formulation and the resulting skin expansion do not by themselves result in the correction of the skin defect, but rather in the generation of excess skin that may be used or manipulated, as described herein, in the further steps of the treatment.

In some embodiments, the sequential injections of the filler formulation comprises a series of increasing amounts (volumes) of formulation from one injection to the next. In accordance with such embodiments, the increase in the amount between one injection to the next may be constant (i.e. the same increase between any two subsequent injections) or may be varied (e.g. wherein the rate of increase varies between each two subsequent injections).

In some embodiments, the sequential injections of the filler formulation comprise a series of equal amounts of formulation injection in each injection event.

In accordance with the present invention, when the skin tissue is a skin tissue from large skin areas the volume of the filler formulation injected per single injection may be between about 20 and 250 cc; when the skin tissue is a skin tissue from small and delicate skin regions, the volume of filler formulation injected per single injection may be between about 10 and 100 cc.

According to methods described herein, the time period between any two subsequent injections may be constant or variable. For example, the injections may be performed exactly every two days or weeks (constant time period), or may be performed at day/week 0, day/week 2, day/week 5, day/week 9 etc (variable time period). The time period between injections may or may not be independent from the amount injected in each injection.

As readily recognized by the skilled artesian, the amount of the filler formulation injected into the skin tissue and the time periods between any two subsequent injections depends on various parameters such as the size of the skin region to be expanded, the type of filler formulation used, the severity of the skin defect to be treated (e.g. size and depth of cut), the location of skin region (e.g. small delicate region such as ear or fingers various large skin area such as abdomen or thighs; hair bearing versus non-hair-bearing skin etc.) and others. Following each injection, the skin expansion may be measured (e.g. once per week) and the amount of filler formulation to be injected in the following injection may be determined based upon the skin expansion measurement and various skin evaluations such as skin color, thickness, pliability, capillary refill, blanching upon pressure, and tightness following a filler formulation injection.

The injection of the filler formulation may be carried out by a medical practitioner or nurse at the patient's home or in the clinic and after preparing the skin tissue for injection (e.g. shaving local hears; using a topical local anesthetic before the injection etc.). The injection of the filler formulation is performed using common means known to the skilled artesian using a syringe and needle size that is suitable for injection into a skin tissue.

In some embodiments, the injection of the filler formulation is performed subcutaneously into the hypodermis. However, the depth of injection may vary based on the filler formulation utilized. For example, it is known that injection of Restylane Touch (500,000 particles/ml, mean particle size 0.2 mm) is recommended to the upper part of the dermis; Restylane (100,000 particles/ml, mean particle size approximately 0.4 mm) should be injected in the mid-part of the dermis; while Restylane Perlane (10,000 particles/ml, mean particle size approximately 0.8 mm) should be injected in the deep layer of the dermis and/or the surface layer of the subcutis. Thus, based on the concentration of the HA particles (particles/ml), the depth of injection may be generally determined.

The injection of the filler formulation can be performed at any location in the skin region to be expanded. Nevertheless, injection in and around the center of the expanded area should be carried out with caution, so as not to puncture an area of maximal tension, such as the top dome of the expanded skin.

In some embodiments, the injection into the skin tissue being adjacent to a skin defect is performed at the periphery of a proposed area of expansion. According to such embodiments, to reduce potential pain, scarring and discomfort of the patient, each injection is performed at a different site in the periphery of the proposed area of expansion.

As readily recognized by the skilled artesian, the duration of injection (e.g. in each separate injection) as well as the gauge size and length of a needle used for the injection, may be dictated by various factors such as skin tension or the patient's tolerance to pain. For example, if the patient cannot tolerate the pain of the injection, the medical practitioner may halt the injection, withdraw the needle and apply mild finger pressure with a gauze dressing for a few minutes before continuing the injection.

The invention further provides a method for therapeutically or cosmetically treating or correcting a skin defect, the method comprising sequentially injecting a filler formulation under a skin tissue, being optionally at a skin region adjacent to the skin defect, to thereby expand and cause growth of the skin tissue and manipulate the grown skin tissue to treat the skin defect.

The “skin region” to be used as a donor skin is generally a section of the human or animal skin, having any size and/or shape, in any area of the body that can be injected with an amount of the filler formulation and which, following expansion and growth, can be used as a donor skin graft. The skin region may be chosen based inter alia on the intended utility of the graft, the skin defect to be corrected, parameters defining suitability of the skin region and other parameters known to a practitioner. In some embodiments, the skin region is a region of a continues large skin area selected from the torso (chest and breasts), abdomen, hips, lower and upper back, buttocks, thighs and calves. In other embodiments, the skin region is from small and delicate skin regions such as the fingers, wrists, elbows, face (including chin, cheeks, forehead and nose), ear, groin, penis and feet.

As detailed herein, injection of the filler formulation into the skin causes continuous stress over a prolonged period of time (e.g. several weeks), resulting in a skin expansion and growth of extra skin (through controlled mechanical overstretch expansion mechanism). The extra skin grown following the filler formulation injection(s) typically matches the color, texture, and thickness of the original surrounding tissue, and thus may be manipulated to cover/grow into/onto (fully or partially) the skin defect. As such, methods of the present invention are particularly useful in treating skin defects in small areas (such as fingertips and nose) that are difficult to treat with other tissue expansion devices (e.g. inflatable balloon expander).

For example, injecting the filler formulation (e.g. sequentially over a period of several weeks) into an abdominal skin tissue that is adjacent to a cut in the abdominal skin causes adjacent skin to gradually expand and grow. After expansion goals are met, surgery may be performed according to procedures known in the field of the art to advance or reconstruct the expanded skin to/over the defect. The remaining filler formulation may be suctioned out from the injected region (e.g. by aspiration though a cannula/needle and/or by injection of hyaluronidase).

Such methods may be used to treat (as means to medically or therapeutically treat a skin condition or a skin disorder) or correct (as means to cosmetically correct a skin defect) a skin defect. The “skin defect” is any type of skin defect which requires skin repair/reconstruction/growth for its healing or cosmetic reconstruction. The skin defect may be a burn (e.g. a flame burn); a cut or laceration (i.e. an irregular tear-like wound caused by e.g. a sharp object which causes the separation of connective tissue elements); a deep cut (i.e. a cut that reveals underlying tissues such as fat, tendon, muscle, or bone); a skin abrasion (e.g. a superficial wound caused by friction or scraping in which the topmost layer of the skin (the epidermis) is scraped off; a skin avulsion (a wound where tissue is not just separated but torn away from the body); a penetration wound (e.g. caused by an object such as a knife entering and coming out from the skin); a gunshot wound (e.g. caused by a bullet or similar projectile driving into or through the body); scars formed following tumor excision or other surgical procedures.

In the context of the present invention, a skin defect does not generally refer to a skin presentation such as wrinkles (e.g. superficial wrinkles), expression lines, or nasolabial folds that may be cosmetically blurred/hidden by using a small volume injection of a polymeric material, such as HA, that does not require substantial lifting capacity. Accordingly, the skin defect, as defined herein is a defect that to be treated requires a substantial lifting capacity of the herein defined filler formulation that is injected in a skin area adjacent to the defect.

Once a desired or sufficient skin expansion is achieved, e.g., in terms of skin area, the excess skin may be used to treat (repair/treat/reconstruct) a skin defect by e.g. extending the skin over the defect and/or harvesting part of the expanded skin and transferring it as an implant to another body region (e.g. by surgery).

In another aspect, the invention provides use of at least one polymer, as defined herein, e.g., HA, in a method of treating at least one or more of skin infections (e.g. post infection skin loss); post traumatic skin loss (e.g. due to friction burns and degloving injuries); deep burns (e.g. deep full-thickness burns); large, open wounds; bed sores or other ulcers on the skin that haven't healed well; skin cancer surgery; pressure ulcers (especially stage IV ulcer which are deep enough to damage underlying muscle and bone); diabetic ulcers; and/or skin loss in the removal of cancerous tissue, the method involves any of the methods of the invention.

In another one of its aspects, the present invention provides a method for preparing a skin graft to repair/treat/reconstruct a skin defect, the method comprising:
sequentially injecting a filler formulation into a skin region to be used as a skin donor, to cause expansion and growth of said skin region to obtain grown skin;
harvesting at least a part of the grown skin to prepare a skin graft; and
implanting the skin graft in a recipient site to treat the skin defect.

In some embodiments, the skin graft is stored under suitable conditions for use at a time of intended implantation. In some embodiments, the graft is implanted in the same subject.

This method of the invention is particularly useful in cases where larger patches of skin grafts/implants are desirable and/or when skin expansion at the site of the skin defect is problematic, for examples in skin regions into which it is difficult to inject.

In accordance with this aspect of the invention, the filler formulation may be injected into an intact skin area (i.e. the donor skin section), e.g., found in regions of the body which contain relatively large patches of intact skin (e.g. abdomen, hips, flank, buttocks, thighs, lower and upper back). The injections are repeated as described herein until skin expansion and growths in the donor skin section is achieved.

In accordance with this aspect of the invention, the injection(s) may be performed at any location of the proposed donor skin section. However, injection at the periphery of the proposed donor skin section may be more suitable in some cases, e.g. when the medical practitioner wishes to avoid puncturing the dome/top of the expanded skin. After expansion is achieved growth of skin in the donor skin section begins and may be later harvested as a skin graft for implantation at a recipient site to treat the skin defect.

For example, for small to medium scalp skin defects (resulting, for example, from trauma or radiation necrosis) a skin graft may be prepared according to a method of the invention by injecting the herein defined filler formulation into a specific donor skin region, e.g., in the abdomen. After validating skin expansion and growth in the donor skin section, a graft is surgically excised out from at least a part of the donor skin section and implanted in the recipient scalp region (e.g. after surgical removal of the damaged skin at the donor section) using any reconstructive surgical technique known in the field of the art that is suitable for reconstruction of skin wounds.

In yet another one of its aspects the present invention provides a skin graft prepared using the steps of:
sequentially injecting a filler formulation into a skin donor section to obtain newly grown skin;
harvesting at least a part of the newly grown skin to be used as a skin graft or to be manipulated or pre-treated to form the skin graft.

The skin graft may be one which can ultimately provide a source of vascular ingrowth for that graft from the vasculature in the recipient skin site.

In some embodiments, the graft is a full-thickness skin graft which contains the epidermis and the complete thickness of dermis from the donor skin area.

In other embodiments, the graft is a split-thickness graft which contain the epidermis and a variable thickness of the dermis.

As readily understood by the medical practitioner, the decision of whether to use full-thickness or split-thickness graft is carried out according to various parameters associated with the recipient and donor skin regions, the type of skin defect etc. For example, grafts with a greater thickness of dermis provide more durable coverage while thin grafts revascularize more efficiently but tend to provide less durable coverage.

In some embodiments, the graft is a meshed graft being a mechanically perforated graft (e.g. in a grid pattern). In accordance with such embodiments, prior to or together with the filler formulation injection into the donor skin site, a synthetic mesh is inserted into the skin and the skin is expanded into the mesh such that newly grown skin covers at least a part of the mesh. The mesh together with the graft are then surgically excised and implanted in the recipient site. The mesh may act as a scaffold for the skin graft and aid the immobilization and revascularization in the recipient skin region.

In accordance with the present invention, the filler formulation may be provided to the e.g. clinic or medical practitioner as a single formulation or as a multicomponent kit wherein the filler formulation is provided in one vial and other components, e.g. saline irrigation solution are provided separately.

Thus, in another one of its aspects, the present invention provides a filler formulation expansion kit comprising:
at least one syringe containing between about 3 to between about 5 ml of the HA filler formulation;
a flask/vial containing a liquid medium or a carrier, e.g., a buffered saline solution;

and instructions of use, e.g., for injection into a skin tissue.

The kit may alternatively contain a vial or a container containing an amount of the filer formulation or of the at least one polymeric material, and a further vial or container which contains a medium. The medium and the polymeric material may be mixed in appropriate amounts as instructed in the kit and delivered into a syringe which may or may not be included in the kit for subsequent delivery by injection.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a photo of a Macrolane injection using 18-gauge needle on a 24-year old woman. The needle entry is at the hair-bearing mastoid area with expansion of the non-hair-bearing skin.

FIG. 2 is an anterolateral view of the same 24-year old woman from FIG. 1 after completion of 20.00 mL Macrolane expansion prior to total right ear reconstruction. The endpoint blanching effect of expansion is noted.

FIGS. 3A-B depict (FIG. 3A) posterior view of a framework from a 33-year old man with the addition of a large cartilage support providing substantial elevation of 1.5 cm projection and (FIG. 3B) an additional view of the framework with the cartilage support.

FIG. 4 is an intraoperative view of left ear partial reconstruction in a 33-year old man demonstrating the Macrolane HA particles within the expanded pocket marked with *. (**) shows the expanded skin surface.

FIG. 5 is an intraoperative view of a total right ear reconstruction in a 24-year old woman showing tensionless redraping of the expanded skin over the projected framework.

FIGS. 6A-H provide preoperative photographs of a 21-year old woman scheduled for total right ear reconstruction using the Marcolane expansion method (A, C, E, G). The new ear had to be set low around the preexisting low set remnant (B, D, F, H). Postoperative view of the patient at 12 month are shown.

FIGS. 7A-H provide preoperative photographs of a 35-year old man scheduled for total right ear reconstruction using the Macrolane expansion method (A, C, E, G). A postoperative view of the patient at 12 month is shown in B, D, F and H.

FIG. 8 is an intraoperative view of a partial right ear reconstruction in a 33-year old man, demonstrating the Macrolane HA particles within the expanded pocket (*) as well as the pliable capsule formed around the HA (**).

FIG. 9 depicts low power (40×) magnification; H&E histology slide of a capsule specimen after 12-week expansion demonstrating an inner layer early capsule with band condensation of collagen (*) surrounding HA filler material (**) associated with a focal inflammatory reaction (***).

FIG. 10 depicts high power (400×) magnification; H&E histology slide of a capsule specimen after 10-week expansion demonstrating fibroblasts (*), macrophages (**), giant histocytes and eosinophils (***) in the vicinity of the HA particles (h).

### DETAILED DESCRIPTION OF EMBODIMENTS

Patients and Methods: A retrospective chart review was performed to identify patients who underwent mastoid skin expansion with Macrolane, followed by total ear reconstruction. Ten patients underwent a one-stage, total or partial ear reconstruction using a rib cartilage framework as described in the modified Nagata technique. Tissue expansion was used in all ear reconstruction cases, either with HA injections or osmotic tissue expanders. Patients were offered both methods. The patients selected for this method were those consecutive cases that were mature enough to tolerate the filler injections and chose the technique. The board of Australian Therapeutic Goods Administration granted approval for the off-label use of Macrolane as an injectable tissue expander for each patient. The Melbourne Institute of Plastic Surgery's ethics board committee approved the study and informed consents were obtained. All data and demographics were collected from patients' charts (Table 1).

TABLE 1
Patients' demographics, expansion data and outcome; Rt (right), Lt (left), F
(female), M (male).

| | | | | Mean | | | | | | | |
|---|---|---|---|---|---|---|---|---|---|---|---|
| | | | | injection | | | | | | Last | |
| | | | | volume | Lowest | Highest | Total | | Ear | follow | |
| | | Expansion | Injection | in one | Volume | volume | volume | Ear | recons. | up | |
| | | duration | session | session | Injected | Injected | Injected | pathology | (total/ | visit, | |
| Age | Gender | (weeks) | number | (ml) | (ml) | (ml) | (ml) | and side | partial) | month | Complication |
| 21 | F | 15 | 8 | 1.94 | 1 | 3 | 15.5 | Rt | total | 24 | Exposed |
| | | | | | | | | Microtia | | | wire on |
| | | | | | | | | | | | antihelix |
| 10 | M | 14 | 7 | 2.07 | 1.5 | 3 | 14.5 | Rt | total | 15 | none |
| | | | | | | | | Microtia | | | |
| 35 | M | 10 | 9 | 1.94 | 1 | 2.5 | 17.45 | Lt | total | 12 | none |
| | | | | | | | | Microtia | | | |
| 33 | M | 11 | 10 | 2.35 | 1.5 | 3 | 23.5 | Acquired | partial | 3 | none |
| | | | | | | | | upper ⅔ | | | |
| | | | | | | | | LT ear | | | |
| | | | | | | | | loss | | | |
| 24 | F | 19 | 13 | 2.3 | 1.5 | 3 | 30 | Rt | total | 6 | none |
| | | | | | | | | Microtia | | | |
| 21 | F | 12 | 10 | 2.13 | 1 | 3 | 21.3 | Rt | total | 12 | none |
| | | | | | | | | microtia | | | |
| 18 | M | 12 | 10 | 1.90 | 1 | 2.5 | 19 | Rt | total | 12 | none |
| | | | | | | | | microtia | | | |
| 14 | M | 10 | 10 | 1.7 | 1 | 3.5 | 17 | Lt | total | 12 | none |
| | | | | | | | | microtia | | | |
| 19 | F | 12 | 10 | 2.00 | 1 | 3 | 20 | Rt | total | 9 | none |
| | | | | | | | | microtia | | | |
| 17 | M | 14 | 10 | 1.94 | 1 | 2.5 | 19.5 | Lt | total | 9 | none |
| | | | | | | | | microtia | | | |

The data was analyzed for complications, revisions and failure rate. In addition, apparent capsule tissue from the expanded pocket of a male patient after 12 weeks of expansion was sent for histological analysis using Hematoxylin and eosin stain (H&E stain).

Non-Surgical Expansion

Complete anterior and posterior three-dimensional framework coverage requires more skin than can be provided by simply utilizing the retroauricular non-hair-bearing skin alone. Nagata has extended the retroauricular mastoid skin flap surface area by including the skin on the posterior aspect of the lobule in most cases. To push the limits further, tissue expanders are placed during a separate surgery, to gain the extra skin necessary for retroauricular definition. In the present study practice Macrolane (VRF 20; NASHA™ gel; Q-Med, Uppsala, Sweden) was used in order to expand the retroauricular mastoid non-hair-bearing skin. The injection technique described herein is consistent with the product's instructions for use.

In the first visit, it is ensures that the patient is prepared to tolerate some discomfort over the course of multiple injections due to the expansion planned. Then the future expansion zone is marked according to the (1) size and measurements of the defect, and (2) location of available non-hair-bearing skin donor site.

On an outpatient basis, serial expansions are performed weekly or every other week. An hour prior to the scheduled procedure, topical local anesthetic cream (23% Lignocaine, 7% Tetracaine) is applied to the mastoid area and is covered with a clean plastic wrap for increased anesthetic efficacy. The procedure then begins with marking of the injection site at the temporal hair bearing area. After prepping the skin and planned injection sites with 70% alcohol, local anesthetic (Xylocaine 1%) is given subcutaneously along the planned injection sites and their tracks. Next, prior to each expansion, several features are examined and recorded. The skin is inspected and gently palpated for assessment of its color, thickness, pliability, capillary refill, blanching upon pressure, and tightness. These evaluations are repeated during the injection procedure several times and serve as the endpoint for an injection session, as traditionally done with tissue expanders.

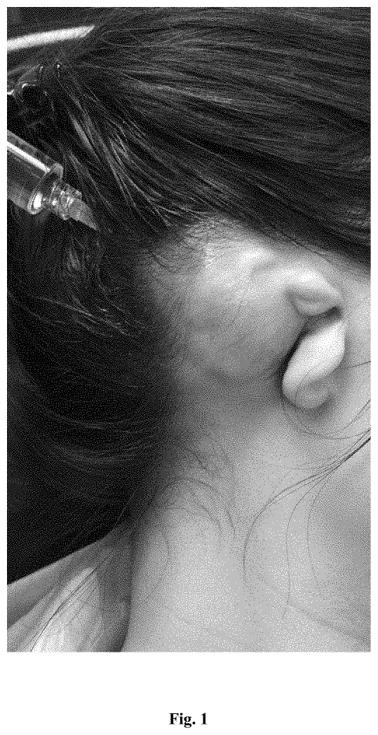

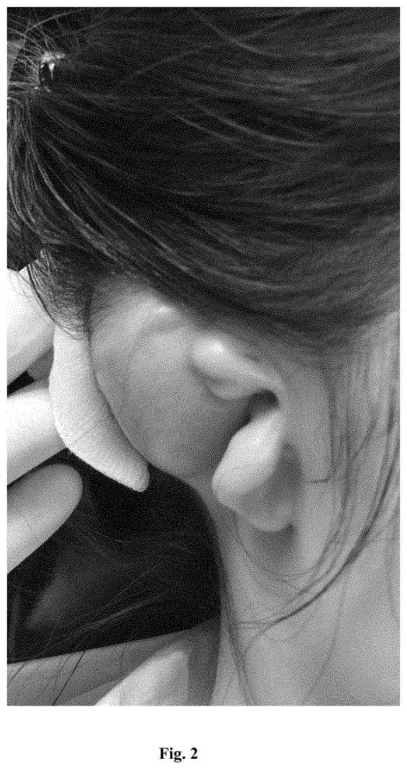

Using an 18-gauge needle, the Macrolane is gradually injected from the marked hair bearing temporal area into the central mastoid area. When an endpoint such as skin tension, blanching or pain is reached, the injection halts, the needle is withdrawn and mild finger pressure with a gauze dressing is applied for a few minutes. One week before the reconstructive surgery, the final session of serial expansions is completed in the office, and a final evaluation of the skin is performed as described above (FIGS. 1-2).

Surgical Technique and Pocket Preparation

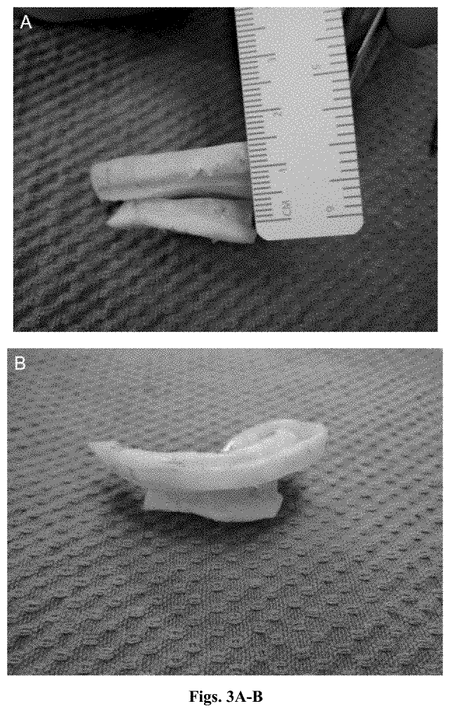

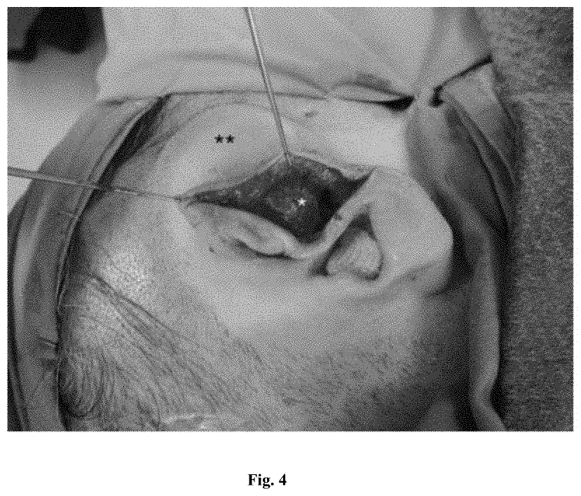

Subsequently, the patient undergoes a costochondral reconstruction per the Nagata method, but with the addition of a large cartilage support providing substantial elevation (FIGS. 3A-B). This technique takes advantage of both the expanded skin pocket and the additional cartilage support to create adequate projection in a single stage. Framework planned location is marked as well as the planned incisions. A rounded W-shaped flap is marked at the mastoid skin that extends onto the posterior surface of the lobule as described by Nagata. The Nagata method is otherwise followed, with preservation of a subcutaneous pedicle and bolster sutures without drains.

Using suction, the Macrolane HA particles are drawn away from the pocket with ease (FIG. 4). Prior to framework insertion, after saline irrigation of the pocket, further undermining is performed beyond the size of the planned reconstructed auricle. Remarkably, in all filler expanded pockets the observed capsule found was thin and pliable and did not require capsulotomy or capsulectomy. This allows preservation of the delayed subdermal plexus, and thus keeps skin vascularity robust.

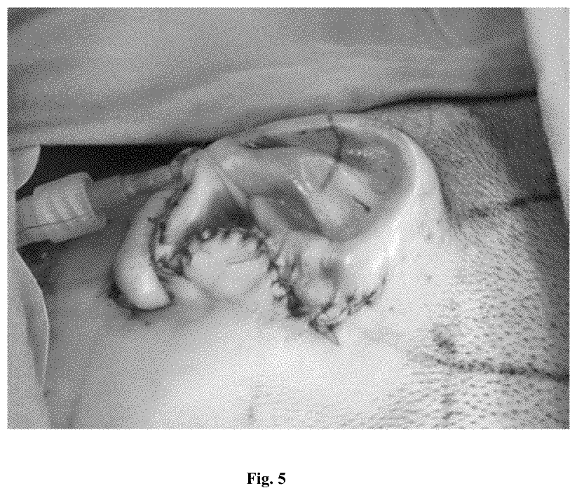

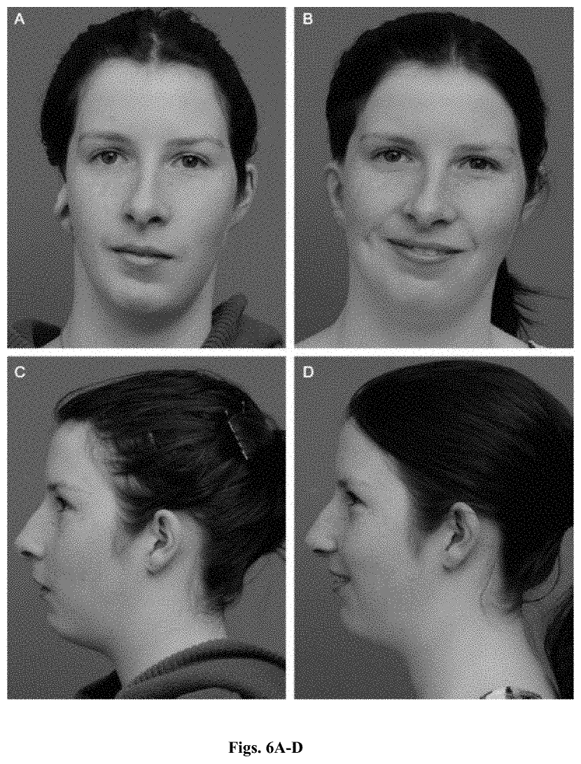

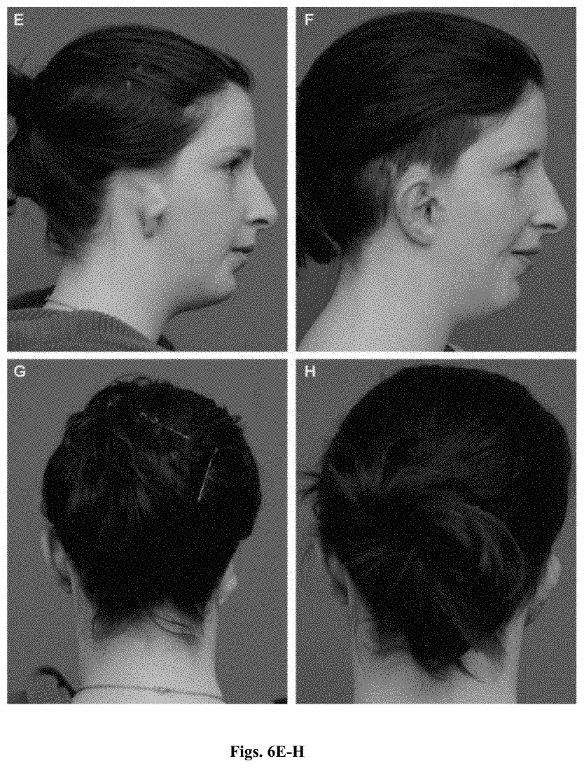

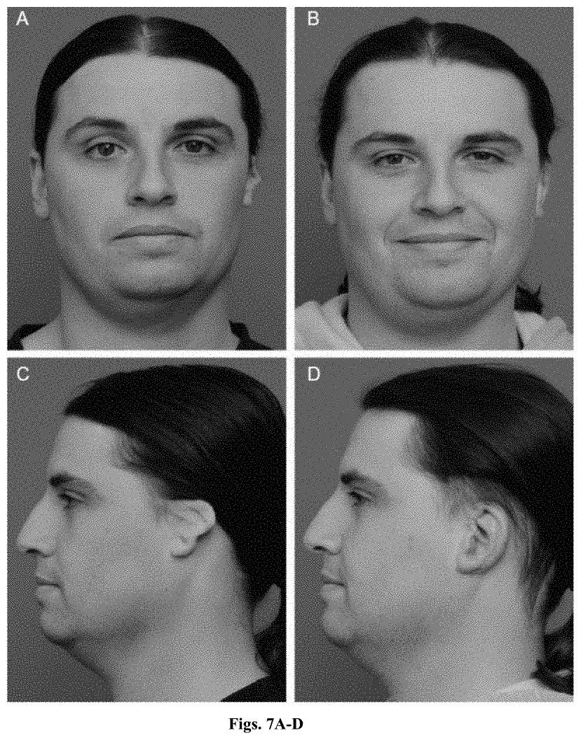

Due to the substantial amount of excess skin generated by expansion, the framework is easily inserted into the skin pocket and rotated into place. The framework is then anchored to the mastoid fascia with 3-0 Prolene sutures (Ethicon, Inc., Somerville, N.J.) superiorly and inferiorly, at the anterior convexity of the helix and the lobule, respectively. A suction drain is placed in vacuum to allow the skin to drape and contour over the framework and then immediately withdrawn at the conclusion of surgery after the dressing is completed. This allows the skin to redrape in a tensionless fashion over the contours of the high relief framework (FIG. 5). Following inset, the incisions are closed with 6-0 nylon interrupted sutures. Betadine soaked Kaltostat (Convatec, Brandon Park, Victoria, Australia) is rolled and placed as bolsters with a covering gauze dressing and a foam ring held with a head net.

Results:

Demographics, data regarding expansion volume, expansion sessions and complications are listed in Table 1. Patients' age ranged from 10 to 35 years (mean age 21.2). Mean follow-up time was 11.1 months (range 3 to 24 months). Nine patients underwent total auricular reconstruction, and one underwent partial reconstruction due to a traumatic upper two-thirds auricular loss. Six cases were right-sided, and four were left-sided. Six were male and four female.

Expansion duration was defined as the time from the first expansion session to the last session, consistently ending one week before surgery. Expansion duration ranged between 10 to 19 weeks (mean 12.9 weeks). Macrolane injection sessions were performed every one to two weeks in accordance with patient and surgeons' timetable and clinical assessment, as previously described for traditional tissue expansion (23). Injection sessions ranged from 7 to 13 meetings (mean 9.7 sessions). The lowest volume of Macrolane injected in one session was 1.0 ml and the highest was 3.5 ml (Table 1). The mean injected volume in one injection session for all patients combined was 2.03 ml (range between 1.70 ml to 2.35 ml). Mean total injected volume per patient was 19.8 ml (range between 14.5 ml to 30.0 ml). No major complications occurred. In 10 patients, only one minor complication was encountered at a 12-month follow up (Table 1). This patient had an exposed wire that was easily removed from the antihelix in the office. At the 6-month clinic follow up visit, patients were interviewed by the senior author. All patients, excluding one that was lost to follow up at 3-month visit, were pleased with their result and expressed their satisfaction with the expansion process regarding pain and comfort, stating that they would do it again (FIGS. 5A-B, 6A-B).

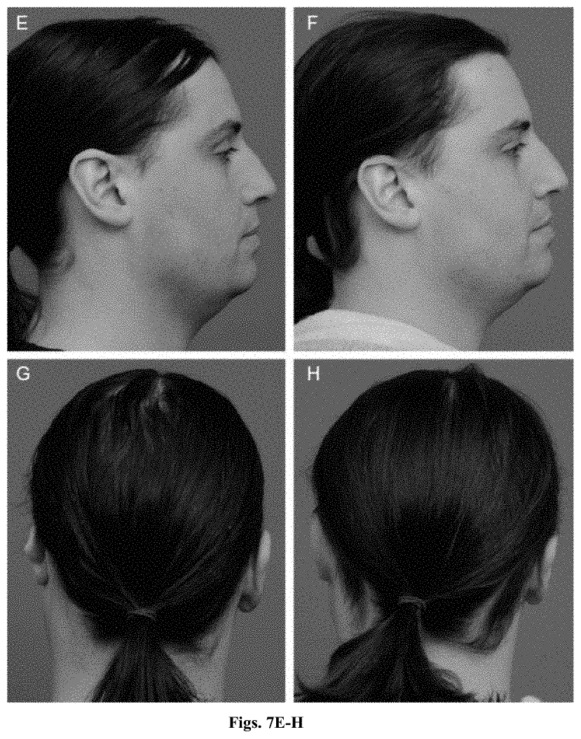

Surrounding tissues from one expanded pocket were sent for Hematoxylin and eosin histological analysis (Melbourne Pathology, Collingwood, VIC, 3066 AUSTRALIA) to better define the capsule that clinically appeared present in all patients. We noticed the capsules were clinically thinner and more pliable than those formed around traditional expanders (FIG. 7). Additionally, no bathtub deformity was encountered on the underside or edges. The thin capsule could easily be manipulated without the need for capsular excision.

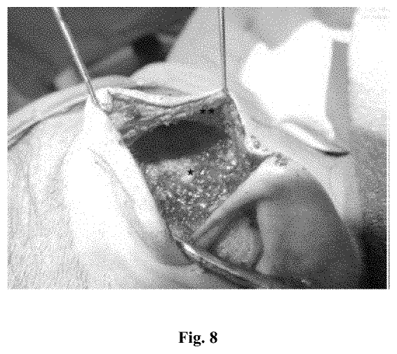

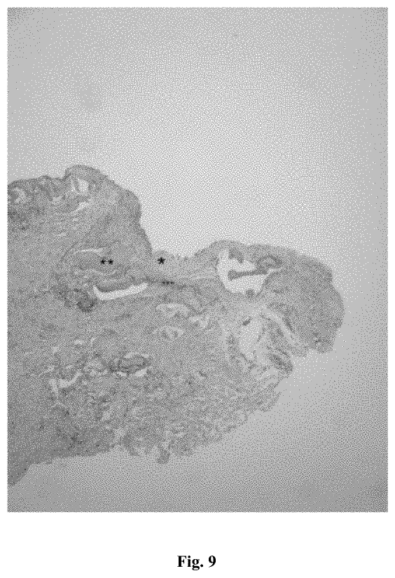

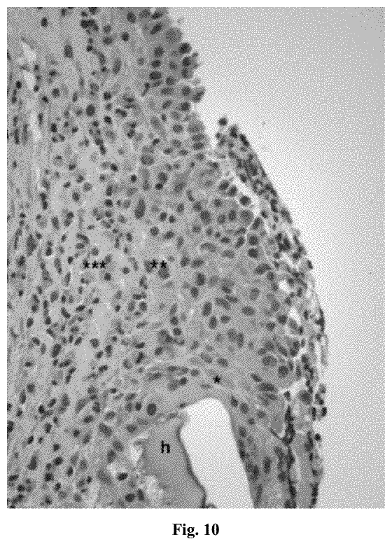

Capsules generally form with an inner layer adjacent to the expander. This layer consists of dense connective tissue (also known as band condensation), fibroblasts, macrophages and eosinophils. An outer capsule layer also develops and is made up of loose connective tissue and abundant vascularization. Histological analysis revealed a capsule surrounding HA filler material associated with a focal inflammatory reaction consisting of fibroblasts, macrophages, giant histiocytes and eosinophils. A thin band condensation was present along the capsule inner layer after 12 weeks of expansion (FIGS. 8-9).

Following Macrolane filler expansion adequate skin was gained for coverage of a fully elevated framework to a height between 1.0 to 1.5 cm. Using this novel non-surgical expansion technique, a fully reconstructed, well-projected, color-matched ear was provided in a single operation without the use of additional skin or fascial flaps.

Auricular reconstruction is both controversial and challenging. Since its introduction by Tanzer in 1959 the costal cartilage framework for autologous reconstruction has become the gold standard for total and some partial auricular reconstruction. Nagata's refinements are well documented and have been adopted by many surgeons. His technique demonstrates exceptional results with fewer complications. Nagata describes a two-stage procedure with incision modifications to allow for increased skin surface area and improved flap vascularity. His description involves framework inset in the first stage, and a second stage surgery with a fascial flap, skin graft and additional rib cartilage for increased projection. Even though Nagatas' two-stage procedure may produce symmetric auricular projection if the contralateral ear is not exceedingly protruded, it does not achieve a deep enough retroauricular sulcus; though, a shadow is created giving the impression of a sulcus. A one-stage, successful elevation of the ear framework with creation of a well-defined retroauricular sulcus continues to be a challenge.

Traditional tissue expansion in auricular reconstruction has been proposed as it may provide more skin coverage of a framework. A thin, color-matching skin pocket can be generated with use of tissue expanders, and the expanded skin can be used alone or with skin or fascial flaps to improve projection. Although some advocate it's use and believe that expansion results in superior outcomes others point out to the adverse effects on the thin skin needed to demonstrate the details of the microtic ear. Some of the major concerns are the added surgical stage for expander insertion and potential complications. For these reasons, tissue expanders have not been widely adopted in ear reconstruction.

Macrolane was used for expansion due to its very large particle size of 1000 gel particles/ml, compared to 100,000 gel particles/mL in Restylane, and 8000-10,000 gel particles/ml in Perlane. The Large particle gels have significant lifting capacity and were therefore thought off as good temporary expansion modality that can substitute traditional tissue expanders. Since the expansion duration typically takes only several months to complete, the substance long term duration of action, efficacy and absorption rate are irrelevant to the expansion modality. Macrolane has been used in Europe and other countries for filling skin contour deformities and buttock enhancement. Previously, it had been used in Europe for breast enhancement, but the absence of controlled clinical trials raised questions regarding its use in the breast. In 2012, after conferring with European authorities, Q-Med stopped marketing Macrolane for breast enhancement. This arose from radiologists' concerns that it makes reading mammograms more difficult, and that HA degradation stimulates neovascularization masking cancer surveillance. Many studies have shown that Macrolane and HA is safe for use in humans, and the concerns regarding its use for breast enhancement did not relate to the safety of the product itself. Due to similar FDA restrictions regarding Macrolane in Australia, we were required to apply individually for each patients' expansion treatment, and receive a distinctive approval. Taking the many advantages of this modality, the Australian Therapeutic Goods Administration authorized the off-label use of Macrolane on an individual basis for tissue expansion in autologous ear reconstruction. As more and more HA products are being FDA approved and added to the U.S market, it can be quite confusing as to which product can give a good lifting capacity and thus an expansion modality. Overall, any product with large particle HA and good lifting capacity such as Juvederm Voluma (Allergan; 2525 Dupont Dr. Irvine, Calif. 92612) and Belotero Balance (Merz Pharmaceuticals, LLC; 4215 Tudor Lane Greensboro, N.C. 27410) would make sense for use.

In the past decade, HA based dermal fillers have provided an attractive and safe nonsurgical alternative to reduce skin wrinkles and folds. Multiple reports show that HA based fillers actually have positive secondary effects on the dermis. Our novel, minimally invasive, off-label use of Macrolane has enabled us to avoid the extra surgical stage required with traditional tissue expanders, while reaping the benefits of expanded skin.

Expansion using HA has many advantages when compared to using traditional tissue expanders. Injections of Macrolane are minimally invasive and performed in the office. This avoids the need for the initial 1^{st} stage surgery and the morbidity associated with additional incisions, including: potential vascular compromise, port problems, expander extrusion, and deflation issues. The repeated Macrolane injections are analogues to the repeated and tolerable injections required to fill a tissue expander, even in the younger age. Filler expansion is performed with a needle entering along the periphery of the proposed area of expansion, unlike traditional expanders where the needle pricks are localized to the port area. This is advantageous as repeated injections in the same area can lead to more scarring and pain. Injection of Macrolane allows the surgeon to adjust the expansion process to each individual patient. The surgeon is not confined to the shapes and boundaries of a traditional expander, or even to a custom made expander. By not being limited to the borders of a traditional expander, the surgeon can better tailor the patient's tissue expansion throughout the entire process. Furthermore, following filler removal, secondary expansion of the expanded skin can be performed in a tailored manner as well. The end point to filler expansion is the skin “give” only and not the expander device's inherent predetermined volume and shape. The complete course of expansion with Macrolane costs substantially less than that of traditional expansion and the added surgical stage fees.

The study of capsule thickness in expanded skin is important and has previously been examined comparing smooth to textured surfaces. Histologic examination of the capsule formed around the Macrolane corroborated our clinical observation of a thin, pliable capsule. While traditional capsules may require surgical manipulation, the capsule found around HA in the herein described study did not require any surgical release or excision, allowing preservation of the vascularity. Capsule thickness in Macrolane expanded skin is an area for further research.

The patient's ages ranged from 10 to 35 years (mean age 21.2). The protocol traditionally has been to wait until 10 years of age or older in all patients as recommended by leaders of modern ear reconstruction. The later age was used in the herein described study for reasons such as minimum chest diameter for sufficient donor material, to avoid a chest deformity, making sure the patient is more emotionally mature, cooperative and involved in the decision to operate. In a study of 10 consecutive patients undergoing single stage autologous ear reconstruction, it was shown that the herein described expansion method was both safe and feasible.

1. A method for skin expansion, the method comprising injecting an amount (or volume) of a filler formulation to a subject at a skin region, said amount being sufficient to cause expansion and growth of skin at said skin region over time, optionally repeating said injection one or more times, to thereby cause expansion and growth of said skin region.

2. A method for therapeutically or cosmetically treating or correcting a skin defect, the method comprising sequentially injecting a filler formulation under at a skin region of a subject, the skin region being optionally a skin region adjacent to the skin defect, to thereby expand and cause growth of a skin tissue at said skin region and manipulate the grown skin tissue to treat the skin defect.

3. The method of claim 1, wherein the method further comprises a step of manipulating said grown skin region to enable use of the skin region in a method of reconstructive treatment or surgery.

4. The method according to claim 1, wherein the filler formulation comprises at least one polymeric material.

5. The method according to claim 4, wherein the at least one polymeric material is selected from poly-L-lactic Acid, polymethylmethacrylate (PMMA), calcium hydroxyapatite (CaHA), chitosan, alginate and hyaluronic acid (HA).

6. The method according to claim 5, wherein the at least one polymeric material is HA.

7. The method according to claim 6, wherein the concentration of HA is at least 5 mg/ml, or is between 5 and 50, 5 and 100, 10 and 50, 10 and 100, 20 and 50, 20 and 100, 30 and 50, 30 and 100mg/ml.

8. The method according to claim 6, wherein the HA is selected from Macrolane®, Hylaform®, Hylaform® Plus, Restylane®, Perlane®, Restylane® Fine Lines, Hylaform° Plus, Captique™, Juvederm™ Ultra, Juvederm™ Ultra plus, Puragen™ Plus, HYAcorp MLF1® and Belotero.

9. The method according to claim 8, wherein the HA in the filler formulation is Macrolane®.

10. The method according to claim 6, wherein the HA is cross-linked HA.

11. The method according to claim 4, wherein the at least one polymeric material is in the form of a phase transfer material.

12. The method according to claim 1, wherein the filler formulation is in a form of material particles.

13. The method according to claim 1, wherein the at least one polymeric material is provided in particulate form.

14. The method according to claim 12, wherein the filler formulation comprises between about 500 and 2,000, 800 to 1,500, or 900 to 1,200 material particles/ml.

15. The method according to claim 1, wherein the filler formulation is in the form of a gel material.

16. The method according to claim 1, wherein the filler formulation further comprises at least one carrier material and further optionally at least one excipient.

17. The method of claim 16, wherein the at least one excipient is selected from the group consisting of a vitamin, a degradation inhibitor, an antioxidant, a stabilizer, a steroid, a retinoid or salt/derivative thereof.

18. The method according to claim 1, wherein the filler formulation is delivered by a single injection or by sequential injections.

19. The method according to claim 1, wherein the volume of filler formulation injected per single injection is between about 20 and 250 cc or between 5 and 100 cc.

20. A method for preparing a skin graft to repair/treat/reconstruct a skin defect, the method comprising:
sequentially injecting a filler formulation into a skin region to be used as a skin donor, to cause expansion and growth of said skin region to obtain grown skin;
harvesting at least a part of the grown skin; and
implanting the harvested skin in a recipient site to treat the skin defect.
